(19) United States
(12) Patent Application Publication
(10) Pub. No.: US 2024/0093183 A1
(43) Pub. Date: Mar. 21, 2024

(54) SYSTEMS AND METHODS FOR MULTIMODAL PROFILING

(71) Applicant: THE TRUSTEES OF COLUMBIA UNIVERSITY IN THE CITY OF NEW YORK, New York, NY (US)

(72) Inventors: Elena E. Kandror, New York, NY (US); Abbas Haider Rizvi, New York, NY (US); Thomas Maniatis, New York, NY (US)

(73) Assignee: THE TRUSTEES OF COLUMBIA UNIVERSITY IN THE CITY OF NEW YORK, New York, NY (US)

(21) Appl. No.: 18/339,789

(22) Filed: Jun. 22, 2023

Related U.S. Application Data
(60) Provisional application No. 63/354,271, filed on Jun. 22, 2022.

(51) Int. Cl. C12N 15/10; C12Q 1/6806

(52) U.S. Cl. CPC C12N 15/1096; C12Q 1/6806

(57) ABSTRACT

The present disclosure relates to the methods and systems for multimodal profiling. The method can include stabilizing a target, performing a tagmentation on the target using an agent, and generating a library using a reverse transcriptase enzyme. The agent can include a Tn5 transposome complex, a Tn5 transposase reagent, or a combination thereof, and the Tn5 transposome complex or the Tn5 transposase reagent can be loaded with an adapter. The reverse transcriptase enzyme can be configured to transcribe DNA from both a DNA template and an RNA template and perform a terminal transferase activity by template switching and an introduction of an adapter sequence into a cDNA and a transposed chromatin of the target.

### CROSS-REFERENCE TO RELATED APPLICATIONS

This application claims priority to the U.S. Provisional Application Ser. No. 63/354,271, filed Jun. 22, 2023, the contents of which are incorporated by reference herein in its entirety.

### BACKGROUND

Certain sequencing techniques (e.g., mcSCRB-Seq, SHARE-Seq) can be used for identifying cellular identity, state, and regulatory mechanisms. However, these techniques can provide a limited depth of coverage and require a large amount of tissue to operate.

Thus, there remain needs for methods and systems for multimodal profiling that can provide increased depth of coverage and efficiency.

### SEQUENCE LISTING

The instant application contains a Sequence Listing which has been submitted electronically in XML format and is hereby incorporated by reference in its entirety. Said XML, copy, created on Aug. 15, 2023, is named 070050.6718_ST26.xml and is 12,664 bytes in size.

### SUMMARY

The disclosed subject matter provides methods, systems, and kits for multimodal profiling. An example method for multimodal profiling can include stabilizing a target, performing a tagmentation on the target using an agent, and generating a library using a reverse transcriptase enzyme. In non-limiting embodiments, the agent can include a Tn5 transposome complex, a Tn5 transposase reagent, or a combination thereof. In non-limiting embodiments, the Tn5 transposome complex or the Tn5 transposase reagent can be loaded with an adapter. In non-limiting embodiments, the reverse transcriptase enzyme can be configured to transcribe DNA from both a DNA template and an RNA template and perform a terminal transferase activity by a template switching and an introduction of an adapter sequence into a cDNA and a transposed chromatin of the target.

In certain embodiments, the adapter can include a first adapter that includes an amino acid sequence at least about 80% identical to the amino acid sequence set forth in SEQ NOs: 1-3. In non-limiting embodiments, the adapter can include a second adapter that includes an amino acid sequence at least about 80% identical to the amino acid sequence set forth in SEQ ID NOs: 4-6.

In certain embodiments, the method can include placing the target on a solution, wherein the solution comprises an effective amount of phosphate-buffered saline (PBS), RNase inhibitor, sodium dodecyl sulphate (SDS), or a combination thereof.

In certain embodiments, the method can further include freezing the target before generating the library.

In certain embodiments, the method can further include performing reverse transcription (RT) on the target to obtain cDNA of the target, performing polymerase chain reaction (PCT) amplification on the cDNA and the chromatin of the target at a predetermined cycle, and splitting the amplified cDNA and the chromatin into a first group for an RNA library and a second group for an assay for Transposase-Accessible Chromatin (ATAC) library. In non-limiting embodiments, a primer for the amplification is introduced at both 5′ and 3′ ends of the first strand of the cDNA and a 3′ end of the chromatin of the target.

In certain embodiments, the RNA library can be generated by performing RNA sequencing on the first group. The first group for the RNA library can include a cell-specific barcode introduced through an RT primer. In non-limiting embodiments, the library can be generated by performing an ATAC sequencing on the second group. The second group for the ATAC library can include a cell-specific barcode introduced through an indexing primer.

In certain embodiments, the reverse transcriptase enzyme can include a Moloney Murine Leukemia Virus (MMLV). In non-limiting embodiments, the target can include nuclei, a whole cell, or a combination thereof. In non-limiting embodiments, the target can be lysated with RNases and stabilized using a solution that irreversibly inactivates RNases.

In certain embodiments, the library is an RNA and ATAC mutual sequencing library (RAM-seq) that simultaneously includes profiles of transcriptome and chromatin accessibility with the target.

An example kit in accordance with the disclosed subject matter can include a stabilizing agent configured to stabilize a target, a tagmentation agent comprising a Tn5 transposome complex, a Tn5 transposase reagent, or a combination thereof, an adapter where the Tn5 transposome complex or the Tn5 transposase reagent can be loaded, and a reverse transcriptase enzyme configured to transcribe DNA from both a DNA template and an RNA template and perform a terminal transferase activity by a template switching and an introduction of an adapter sequence into a cDNA and a transposed chromatin of the target for generating a library.

In certain embodiments, the adapter can include a first adapter that includes an amino acid sequence at least about 80% identical to the amino acid sequence set forth in SEQ ID NOs: 1-3. In non-limiting embodiments, the adapter can include a second adapter that includes an amino acid sequence at least about 80% identical to the amino acid sequence set forth in SEQ ID NOs: 4-6.

In certain embodiments, the kit can further include a primer for the amplification of the cDNA of the target. The primer can be configured to be introduced at both 5′ and 3′ ends of the first strand of the cDNA and a 3′ end of chromatin of the target.

In certain embodiments, the reverse transcriptase enzyme can include a Moloney Murine Leukemia Virus (MMLV) reverse transcriptase. In non-limiting embodiments, the target can include nuclei, a whole cell, or a combination thereof. In non-limiting embodiments, the stabilizing agent can be configured to stabilize the target by irreversibly inactivating RNases.

In certain embodiments, the library can be an RNA and ATAC mutual sequencing library (RAM-seq) that simultaneously includes profiles of transcriptome and chromatin accessibility with the target.

### BRIEF DESCRIPTION OF THE DRAWINGS

The subject matter of the application will be more readily understood from the following detailed description when read in conjunction with the accompanying drawings in which:

FIG. 1 provides a diagram showing an example method for multimodal profiling in accordance with the disclosed subject matter (SEQ ID NOs. 7-8 top to bottom respectively).

FIG. 2 provides graphs showing example profiles obtained by performing the disclosed mutual sequencing in accordance with the disclosed subject matter.

The accompanying drawings, which are incorporated in and constitute part of this specification, are included to illustrate and provide a further understanding of the devices of the disclosed subject matter. Together with the description, the drawings serve to explain the principles of the disclosed subject matter.

### DETAILED DESCRIPTION OF THE INVENTION

The disclosed subject matter provides techniques for multimodal profiling. The disclosed techniques can utilize simultaneous Ribonucleic Acid (RNA)/Assay for Transposase-Accessible Chromatin using sequencing (ATAC) profiling that can interrogate cellular heterogeneity and biological complexity via gene expression profiles and gene regulatory programs. The disclosed multimodal interrogation of RNA and chromatin accessibility can provide insight into cellular identity, state, and regulatory underpinnings.

Non-limiting embodiments of the present disclosure are described by the present specification and Examples.

### Definitions

Unless defined otherwise, all technical and scientific terms used herein have the meaning commonly understood by a person skilled in the art to which this disclosed subject matter belongs. The following references provide one of skill with a general definition of certain terms used in this disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

An “individual” or “subject” herein is a vertebrate, such as a human or non-human animal, for example, a mammal. Mammals include, but are not limited to, humans, non-human primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

An “effective amount” or “therapeutically effective amount” is an amount effective, at dosages and for periods of time necessary, that produces a desired effect.

By “increase” is meant to alter positively by at least about 5%. A positive alteration can be an increase of about 5%, about 10%, about 25%, about 30%, about 50%, about 75%, about 100% or more.

By “reduce” is meant to alter negatively by at least about 5%. A negative alteration can be a decrease of about 5%, about 10%, about 25%, about 30%, about 50%, about 75% or more, even by about 100%.

The terms “nucleic acid sequence” and “polynucleotide,” as used herein, refer to a single or double-stranded covalently-linked sequence of nucleotides in which the 3′ and 5′ ends on each nucleotide are joined by phosphodiester bonds. The polynucleotide can include deoxyribonucleotide bases or ribonucleotide bases and can be manufactured synthetically in vitro or isolated from natural sources.

The terms “polypeptide,” “peptide,” “amino acid sequence,” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

The term “dosage” is intended to encompass a formulation expressed in terms of total amounts for a given timeframe, for example, as μg/kg/hr, μg/kg/day, mg/kg/day, or mg/kg/hr. The dosage is the amount of an ingredient administered in accordance with a particular dosage regimen. A “dose” is an amount of an agent administered to a mammal in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning, in any case, will be apparent from the context.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. Ranges disclosed herein, for example, “between about X and about Y” are, unless specified otherwise, inclusive of range limits about X and about Y as well as X and Y. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50 to 30, 50 to 20, and 50 to 10 in the other direction.

Methods for Multimodal Profiling

In certain embodiments, the disclosed subject matter provides methods of ribonucleic acid (RNA) and assay for transposase-accessible chromatin (ATAC) mutual sequencing (RAM-seq). The RAM-seq can be a multiplexed technique to simultaneously profile the transcriptome and chromatin accessibility within a single cell (e.g., at high depth). The disclosed RAM-seq can be scalable to large numbers of cells and enables higher coverage of the transcriptome (e.g., up to about 8,000 genes detected per cell with an average of 40,000 unique transcripts per cell) and chromatin accessibility (e.g., an average of about 3,500 unique chromatin fragments detected per cell with about 60% fraction of reads in peak) as compared to certain other measurement pipelines at an equivalent cost.

In certain embodiments, the method can include stabilizing a target. In non-limiting embodiments, the target can include monodispersed nuclei or whole cells. For example, monodispersed nuclei or whole cells can be stabilized using RNAsecure. In non-limiting embodiments, the target can be lysated by RNase, and the stabilizing agent (e.g., RNAsecure) can irreversibly inactivate RNases.

In certain embodiments, the method can include performing a tagmentation with the stabilized target. For example, the monodispersed nuclei or whole cells can be subjected to bulk tagmentation using any Tn5 transposome complex or Tn5 transposase reagent loaded with either regular (e.g., commercially preloaded) adapters or custom adapters. In non-limiting embodiments, The Tn5 transpose reagent can include any Tn5 transposase (e.g., bacterial Tn5 transposase, hyperactive Tn5 transposase, enzymatic Tn5 transposase, or combinations thereof).

In certain embodiments, the regular adapter can include an amino acid sequence at least about 80% identical to the following amino acid sequence set forth in SEQ ID NOs. 1-3.

A:
(SEQ ID NO: 1)
TCGTCGGCAGCGTCAGATGTGTATAAGAGACAG
B:
(SEQ ID NO: 2)
GTCTCGTGGGCTCGGAGATGTGTATAAGAGACAG
R:
(SEQ ID NO: 3)
/5Phos/CTGTCTCTTATACACATCT

In certain embodiments, the customized adapter can include an amino acid sequence at least about 80% identical to the following amino acid sequence set forth in SEQ ID NOs: 4-6.

A:
(SEQ ID NO: 4)
ACACTCTTTCCCTACACGACGCTCGTCGGCAGCGTCAGATGTGTATAAGA
GACAG
B:
(SEQ ID NO: 5)
ACACTCTTTCCCTACACGACGCGTCTCGTGGGCTCGGAGATGTGTATAAG
AGACAG
R:
(SEQ ID NO: 6)
/5Phos/CTGTCTCTTATACACATCT

In certain embodiments, the method can include depositing the target (e.g., a single tagmented nuclei or whole cells) into an individual well. The individual well can include a solution. In non-limiting embodiments, the solution can include phosphate-buffered saline (PBS), RNase inhibitor, and/or sodium dodecyl sulphate (SDS). For example, the single tagmented nuclei or whole cells can be dispositioned into individual wells of a microwell plate (e.g., FACS sorted into a 384 well plate) containing 1 ul of either PBS+RNase inhibitor (e.g., PBS+0.2 U/ul Superasin) or 0.2% SDS.

In non-limiting embodiments, the method can include freezing the target before generating a library. For example, the plates, including targets, can be frozen in liquid nitrogen and stored at −80C for up to 6 months prior to library generation.

In certain embodiments, the method can include generating a RAM-seq library using the target. The disclosed RAM-seq library generation can leverage both the ability of reverse transcriptase to transcribe DNA off of both a DNA and RNA template and the terminal transferase activity of reverse transcriptase. This can provide for template switching and introduction of adapter sequences into the cDNA and transposed chromatin.

In non-limiting embodiments, any reverse transcriptase enzyme can be applicable to the disclosed RAM-seq methods. For example, Maxima H minus, which can provide efficient thermal stability and processivity, can be used for the disclosed RAM-seq, allowing for the capture of full-length transcriptome products.

In certain embodiments, the method can include performing Reverse transcription (RT). For example, RT can be performed with the modifications of excluding Proteinase K and crowding agents and decreasing the reaction volume (5 ul/well). In non-limiting embodiments, a first-strand cDNA can include a custom second-round amplification sequence, a well-specific barcode, and/or a unique molecular identifier. Both cDNA and tagmented chromatin (e.g., extended from the tagmentation product 5′ overhang) can include a 3′ adapter sequence introduced via template switching. This adapter sequence can be used as a handle for in-well simultaneous single primer polymerase chain reaction (PCR) amplification of cDNA and tagmented chromatin. In some embodiments, DNA polymerase can be used in this amplification. For example, KAPA HiFi DNA Polymerase can be used. In non-limiting embodiments, after first-round PCR amplification, a predetermined amount (e.g., half) of the reaction can be transferred into a second microwell plate for ATAC library generation. The remaining (e.g., half) can be pooled for transcriptome library generation.

In certain embodiments, the pooled cDNA can be cleaned up and concentrated on beads (e.g., commercially available Ampure beads or homemade beads). The cDNA can be tagmented and undergo second-round amplification and indexing (e.g., following the Illumina Nextera XT protocol) using a P5Next primer (mcSCRB), which can selectively amplify cDNA over the tagmented chromatin product and an Illumina N7xx primer.

In certain embodiments, the single-cell ATAC libraries can be barcoded by second-round PCR amplification in-well using dual indexing primers recognizing the transposase adapters (e.g., expanded indexes based on Illumina N7xx and S5xx primers), which can selectively amplify ATAC product over cDNA. Barcoded ATAC libraries can be pooled, cleaned up, and concentrated on beads. The eluted ATAC library can further be amplified using primers against sequencing adapters present in the second-round amplification primers. In non-limiting embodiments, the disclosed library generation for RAM-seq can be designed or adjusted for various sequencing platforms (e.g., Ion PGM, PacBio RS, and/or Illumina sequencing). For example, primer and adapter sequences can be adjusted for alternate sequencing platforms (e.g., Ion PGM, PacBio RS, and/or Illumina sequencing). In non-limiting embodiments, the disclosed technique can be used for detecting chromosomal interactions and RNA, plate-based assays for chromatin accessibility, and any tagmentaion related workflows.

In certain embodiments, the disclosed technique can provide for jointly profiling a relatively few numbers of cells, e.g., 96 cells, making it a valuable resource when input material is limited, the elimination of a need for third-party library preparation instruments as the protocol can be performed in microwell plates, retention of >90% of cells passing stringent quality control (QC) filtering including doublet discrimination, library complexity, and mitochondrial contamination,) optional inclusion of the External RNA Controls Consortium (ERCC) spike-ins allowing for downstream analytical pipelines, and/or the elimination of a need for chemical or physical partitioning prior to amplification.

The disclosed techniques can allow simultaneous profiling of chromatin accessibility with RNA measurements in addition to RNA readout. The disclosed techniques can provide a higher depth of coverage for RNA and chromatin accessibility compared to certain techniques (e.g., chromium single-cell multiome ATAC+gene expression). The post-sequencing mapping efficiency and cell assignment can be higher than Chromium, decreasing sequencing costs.

The disclosed technique can provide an improved depth of coverage for RNA. For example, the disclosed techniques can be performed without relying on the ligation of adapters or split-pool strategy, resulting in more cells passing QC at more stringent filtering conditions and more reads being mapped and assigned to cells that pass QC (thus decreasing sequencing costs).

The disclosed RAM-seq can provide an affordable and convenient way to simultaneously profile the transcriptome and chromatin accessibility in single cells. Furthermore, RAM-seq can outperform existing technologies by generating higher quality data and has the flexibility to be scaled to large numbers of cells or be applied to biological directions where tissue is limiting.

Kits

The present disclosure provides kits for multimodal profiling. The kit for multimodal profiling can include a stabilizing agent configured to stabilize a target, a tagmentation agent that can include a Tn5 transposome complex, a Tn5 transposase reagent, or a combination thereof, an adapter, and a reverse transcriptase enzyme.

In certain embodiments, the reverse transcriptase enzyme can be configured to transcribe DNA from both a DNA template and an RNA template and perform a terminal transferase activity by a template switching and an introduction of an adapter sequence into a cDNA and a transposed chromatin of the target for generating a library. In non-limiting embodiments, the reverse transcriptase enzyme can include a Moloney Murine Leukemia Virus (MMLV) reverse transcriptase.

In certain embodiments, the kit can include an adapter that can carry the Tn5 transposome complex or the Tn5 transposase reagent. The adaptor can be a regular adaptor or a customized adaptor. In non-limiting embodiments, the regular adapter can include an amino acid sequence at least about 80% identical to the amino acid sequence set forth in SEQ ID Nos: 1-3. In non-limiting embodiments, the customized adapter can include an amino acid sequence at least about 80% identical to the amino acid sequence set forth in SEQ ID NOs: 4-6.

In certain embodiments, the kit can further include a primer for the amplification of cDNA and/or chromatin of the target. For example, the primer can be configured to be introduced at both 5′ and 3′ ends of the first strand of the cDNA and a 3′ end of chromatin of the target.

In certain embodiments, the target can include nuclei, a whole cell, or a combination thereof. The target can be lysated with RNases for the disclosed multimodal profiling, and the stabilizing agent can irreversibly inactivate the RNases after lysating the target.

In certain embodiments, the disclosed kit and components can be used with any sequencing devices available in the art.

The description herein merely illustrates the principles of the disclosed subject matter. Various modifications and alterations to the described embodiments will be apparent to those skilled in the art in view of the teachings herein. Further, it should be noted that the language used herein has been selected for readability rather than to delineate or limit the disclosed subject matter. Accordingly, the disclosure herein is intended to be illustrative, but not limiting, of the scope of the disclosed subject matter.

### EXAMPLES

The present disclosure will be better understood by reference to the following Examples, which are provided as exemplary of the presently disclosed subject matter and not by way of limitation.

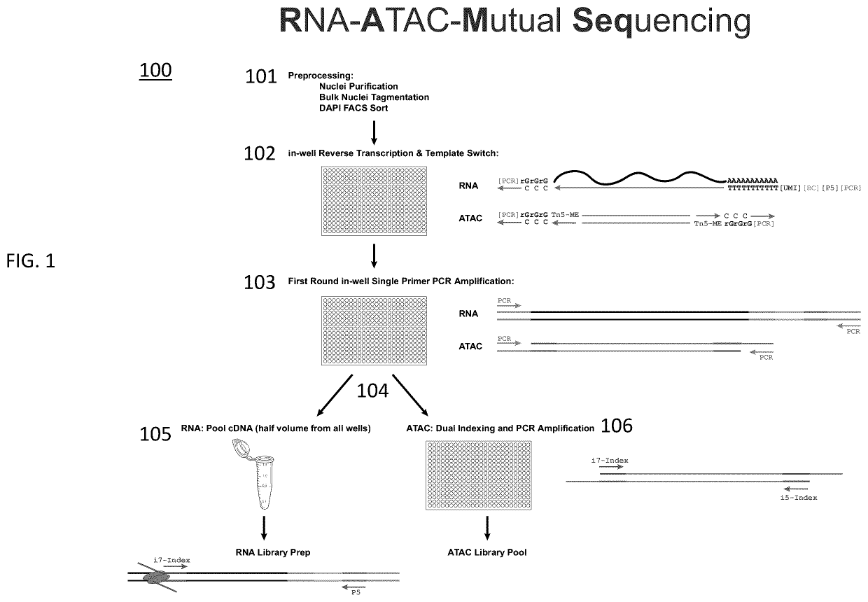

The disclosed RNA and ATAC Mutual Sequencing (RAM-seq) techniques 100 can provide simultaneous profiling of the transcriptome and chromatin accessibility. As shown in FIG. 1, the target cell was preprocessed through various assays (e.g., Nuclei Purification, Bulk Nuclei Tagmentation, DAPI FACS sorting, or combinations thereof) 101, depending on the purposes and the types of targets. The disclosed techniques were applicable to both in-well amplification (e.g., for limited cell numbers) and droplet-based methods. Input into RAM-seq was tagmented nuclei or tagmented whole cells. RAM-seq was performed using the inherent terminal transferase and template switching capability of a Moloney Murine Leukemia Virus (MMLV) reverse transcriptase. During reverse transcription (RT) 102, a template for single primer amplification was introduced at both the 5′ and 3′ ends of the first strand cDNA (allowing for exponential amplification) and at the 3′ end of the tagmented chromatin (allowing for linear amplification). An initial single primer amplification PCR 103 was performed at a predetermined cycle, followed by reaction splitting for RNA and ATAC library preparation 104. For RNA library preparation 105, samples were pooled, purified, tagmented, and selectively amplified using a common i5 Illumina primer and pool-specific i7 Illumina primer (allowing for multiple plate multiplexing). The cell-specific barcode for RNA libraries was introduced via the RT primer. For ATAC library preparation 106, selective amplification and cell-specific barcoding were accomplished via Illumina i5 and i7 dual indexing in a second round amplification reaction, followed by pooling and purification. Illumina amplification and indexing primer sequences can be replaced by custom sequences without adverse effects on data quality. RNA sequencing and ATAC sequencing can be performed by techniques available in the art.

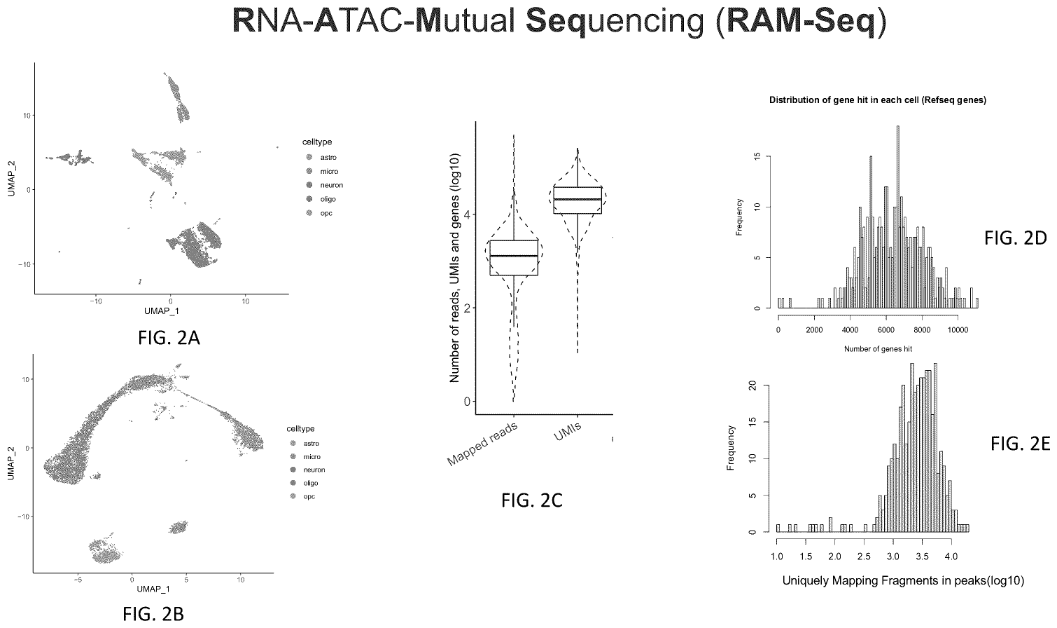

As shown in FIGS. 2A-2E, due to the single primer pre-amplification process that simultaneously produces reads for both RNA and ATAC, the depth of detection in RNA space (UMIs per cell, and genes detected per cell) can be equivalent to traditional high-depth RNA-seq methods, and outperform existing simultaneous measurements of nuclei by about ˜5 fold. The common metrics for ATAC data quality (reads per cell and fraction of reads in peaks) outperform traditional simultaneous measurements about ˜2-fold. This is a substantial improvement in both modalities and allows for significantly improved sensitivity in downstream analysis. Furthermore, in-well RAM-seq recovers ˜95% of input cells passing stringent QC, as opposed to droplet-based methods, which recover 40-65% of input cells, giving the user tight control over input material. This is especially important when the research focuses on a subpopulation of cells with limited input availability.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments described in the specification.

Patents, patent applications, publications, product descriptions and protocols are cited throughout this application, the disclosures of which are incorporated herein by reference in their entirety for all purposes.

1. A method for multimodal profiling comprising:
stabilizing a target;
performing a tagmentation on the target using an agent, wherein the agent comprises a Tn5 transposome complex, a Tn5 transposase reagent, or a combination thereof, wherein the Tn5 transposome complex or the Tn5 transposase reagent is loaded with an adapter; and
generating a library using a reverse transcriptase enzyme, wherein the reverse transcriptase enzyme is configured to transcribe DNA from both a DNA template and an RNA template and perform a terminal transferase activity by a template switching and an introduction of an adapter sequence into a cDNA and a transposed chromatin of the target.

2. The method of claim 1, wherein the adapter comprises a first adapter that includes an amino acid sequence at least about 80% identical to the amino acid sequence set forth in SEQ ID NOs: 1-3.

3. The method of claim 1, wherein the adapter comprises a second adapter that includes an amino acid sequence at least about 80% identical to the amino acid sequence set forth in SEQ ID NOs: 4-6.

4. The method of claim 1, further comprising placing the target on a solution, wherein the solution comprises an effective amount of phosphate-buffered saline (PBS), RNase inhibitor, sodium dodecyl sulphate (SDS), or a combination thereof.

5. The method of claim 1 further comprising freezing the target before generating the library.

6. The method of claim 1, further comprising performing reverse transcription (RT) on the target to obtain cDNA of the target, performing polymerase chain reaction (PCT) amplification on the cDNA and the chromatin of the target at a predetermined cycle, wherein a primer for the amplification is introduced at both 5′ and 3′ ends of a first strand of the cDNA and a 3′ end of the chromatin of the target; and
splitting the amplified cDNA and the chromatin into a first group for an RNA library and a second group for an assay for Transposase-Accessible Chromatin (ATAC) library.

7. The method of claim 6, wherein the generating the library comprises
generating the RNA library by performing an RNA sequencing on the first group, wherein the first group for the RNA library comprises a cell-specific barcode introduced through an RT primer; and
generating the ATAC library by performing an ATAC sequencing on the second group, wherein the second group for the ATAC library comprises a cell-specific barcode introduced through an indexing primer.

8. The method of claim 1, the reverse transcriptase enzyme comprises a Moloney Murine Leukemia Virus (MMLV)

9. The method of claim 1, wherein the target comprises a nucleus, a whole cell, or a combination thereof.

10. The method of claim 1, wherein the target is lysated with RNases and stabilized using a solution that irreversibly inactivates RNases.

11. The method of claim 1, wherein the library is an RNA and ATACT mutual sequencing library (RAM-seq) that simultaneously includes profiles of transcriptome and chromatin accessibility with the target.

12. A kit for multimodal profiling comprising
a stabilizing agent configured to stabilize a target;
a tagmentation agent comprising a Tn5 transposome complex, a Tn5 transposase reagent, or a combination thereof,
an adapter, wherein the Tn5 transposome complex or the Tn5 transposase reagent is loaded with an adapter; and
a reverse transcriptase enzyme configured to transcribe DNA from both a DNA template and an RNA template and perform a terminal transferase activity by a template switching and an introduction of an adapter sequence into a cDNA and a transposed chromatin of the target for generating a library.

13. The kit of claim 12, wherein the adapter comprises a first adapter that includes an amino acid sequence at least about 80% identical to the amino acid sequence set forth in SEQ ID NOs: 1-3.

14. The kit of claim 12, wherein the adapter comprises a second adapter that includes an amino acid sequence at least about 80% identical to the amino acid sequence set forth in SEQ ID NOs: 4-6.

15. The kit of claim 12, further comprising a primer for amplification of cDNA of the target, wherein the primer is configured to be introduced at both 5′ and 3′ ends of a first strand of the cDNA and a 3′ end of a chromatin of the target.

16. The kit of claim 12, the reverse transcriptase enzyme comprises a Moloney Murine Leukemia Virus (MMLV) reverse transcriptase.

17. The kit of claim 12, wherein the target comprises a nucleus, a whole cell, or a combination thereof.

18. The kit of claim 12, wherein the stabilizing agent is configured to stabilize the target by irreversibly inactivating RNases.

19. The kit of claim 1, wherein the library is an RNA and ATAC mutual sequencing library (RAM-seq) that simultaneously includes profiles of transcriptome and chromatin accessibility with the target.


SEQUENCE LISTING (8 sequences)

SEQ ID NO: 1 - DNA, 33 bp, synthetic construct
  misc_feature 1..33: note = Description of sequence: Adapter component
  source 1..33: mol_type = other DNA
tcgtcggcagcgtcagatgtgtataagagacag

SEQ ID NO: 2 - DNA, 34 bp, synthetic construct
  misc_feature 1..34: note = Description of sequence: Adapter component
  source 1..34: mol_type = other DNA
gtctcgtgggctcggagatgtgtataagagacag

SEQ ID NO: 3 - DNA, 19 bp, synthetic construct
  misc_feature 1..19: note = Description of sequence: Adapter component
  source 1..19: mol_type = other DNA
  modified_base 1: mod_base = OTHER; note = 5' phosphate group
ctgtctcttatacacatct

SEQ ID NO: 4 - DNA, 55 bp, synthetic construct
  misc_feature 1..55: note = Description of sequence: Adapter component
  source 1..55: mol_type = other DNA
acactctttccctacacgacgctcgtcggcagcgtcagatgtgtataagagacag

SEQ ID NO: 5 - DNA, 56 bp, synthetic construct
  misc_feature 1..56: note = Description of sequence: Adapter component
  source 1..56: mol_type = other DNA
acactctttccctacacgacgcgtctcgtgggctcggagatgtgtataagagacag

SEQ ID NO: 6 - DNA, 19 bp, synthetic construct
  misc_feature 1..19: note = Description of sequence: Adapter component
  source 1..19: mol_type = other DNA
  modified_base 1: mod_base = OTHER; note = 5' phosphate group
ctgtctcttatacacatct

SEQ ID NO: 7 - RNA, 10 nt, synthetic construct
  misc_feature 1..10: note = Description of sequence: RNA Poly A sequence
  source 1..10: mol_type = other RNA
aaaaaaaaaa

SEQ ID NO: 8 - DNA, 10 bp, synthetic construct
  misc_feature 1..10: note = Description of sequence: Reverse transcript of Poly A sequence
  source 1..10: mol_type = other DNA
tttttttttt